(19) United States
(12) Patent Application Publication
(10) Pub. No.: US 2009/0220577 A1
(43) Pub. Date: Sep. 3, 2009

(54) Moulded body for medically treating wounds

(76) Inventors: Helmut Hierlemann, Göppingen (DE); Heicrich Planck, Nürtingen (DE); Christian Uhlig, Stuttgart (DE)

Correspondence Address: THE NATH LAW GROUP, 112 South West Street, Alexandria, VA, 22314, US

(21) Appl. No.: 11/991,315

(22) PCT Filed: Aug. 30, 2006
(86) PCT No.: PCT/EP2006/008470

§ 371 Date: Jan. 22, 2009

(30) Foreign Application Priority Data
Sep. 1, 2005 (DE) 10 2005 042 707.3

(51) Int. Cl. A61K 9/70; A61P 17/02

(52) U.S. Cl. 424/443

(57) ABSTRACT

The invention relates to an absorbable and porous shaped article for the medical treatment of wounds, in particular large, deep wounds with heavy discharge, where the shaped article is in the form of a foam structure which includes a co- and/or terpolymer based on the monomers lactide, triethylene carbonate, ε-caprolactone and/or dioxan-2-one.

The present invention relates to an absorbable and porous shaped article for the medical treatment of wounds, especially as wound-contact material, to a process for the production thereof, and to various uses of the shaped article.

There has been a steady increase in recent years in the clinical need for possible treatments for acute and chronic wounds, especially by occlusion with suitable covering materials. In particular chronic wounds, for example venous ulcers, diabetically induced ulcers of the extremities, and pressure ulcers, the prevalence of which is continuously increasing in western industrialized countries, often mean for the patient a risk of serious infection, a long hospitalization time and possibly even amputation of the affected limbs. However, the treatment of acute wounds, for example burn wounds, also imposes high demands on the treatment of the traumatized body regions in order to avoid undesired risks, in particular loss of water, fall in temperature and an increased risk of infection.

For this purpose, a large number of biocompatible dressing materials based on natural and synthetic substances have been developed. The dressing materials currently commercially available consist predominantly of nonabsorbable synthetic polymers, for example silicone, polyurethane (PU), polypropylene (PP), polyethylene terephthalate (PET), polyamide (PA) and polytetrafluoroethylene (PTFE), or of absorbable polymers of natural origin, especially collagen, hyaluronic acid, cellulose, polylactide or polyglycolide.

The disadvantages of the known materials consist in particular of their incomplete or at least very slow absorbability in the patient's body. In addition, unwanted adhesions of the dressing materials to the tissue of the body may occur and may induce a painful traumatization of the patient when a surface dressing is changed. In addition, detachment of freshly epithelized areas of skin may lead to formation of scars and thus to an unsatisfactory cosmetic result.

DE 100 41 684 A1 has disclosed an efficient coating polymer based on lactide, trimethylene carbonate and ε-caprolactone which is used in particular for the treatment of first and second degree burn wounds. The coating material exhibits for example advantageous degradation and absorption times in vivo so that in particular an accumulation of foreign materials and, connected therewith, unpredictable side effects in the relevant person's body are avoided.

However, the problem which frequently arises with ulcers, i.e. large-area, deep wounds with heavy discharge, is that relatively large amounts of wound fluid collect in the wound bed or environment, thus increasing the risk of a spreading infection owing to the infectious material accumulated in the wound fluid. This entails uncalculable risks of the treatment for the patient and is therefore undesired from the medical viewpoint. In addition, with areas of the body with particularly heavy exudation there is always an increased risk of infection with further pathogens.

The object of the invention is to provide a shaped article which is made of a biocompatible polymer and which overcomes the problems known from the prior art and in particular allows large amounts of infectious wound fluid to be removed rapidly and thus enables an efficient possibility for the treatment and management of large-area, deep wounds with heavy discharge, for example associated with ulcers and/or deep dermal burns, especially second and/or third degree burns.

This object is achieved by an absorbable and porous shaped article for the medical treatment of wounds, in particular as wound-contact material, characterized in that it is in the form of a foam structure which includes a co- and/or terpolymer based on the monomers lactide, trimethylene carbonate, ε-caprolactone and/or dioxan-2-one. The dioxan-2-one monomer is preferably p-dioxan-2-one.

The absorbable and porous shaped article preferably takes the form of a foam structure which includes a terpolymer based on the monomers lactide, trimethylene carbonate and ε-caprolactone, and the foam structure preferably consists of this terpolymer. The terpolymer preferably has a content of lactide not exceeding 85% by weight, in particular not exceeding 80% by weight, of trimethylene carbonate in the range from 5 to 20% by weight, in particular 10 to 20% by weight, and of ε-caprolactone in the range from 5 to 20% by weight, in particular 5 to 15% by weight. For further details, reference is made to DE 100 41 684 A1, the disclosure of which is expressly intended to be encompassed by the present invention.

The lactide/trimethylene carbonate/ε-caprolactone monomers may be present in particular in ratios in the range from 88/8/4 to 70/20/10% by weight based on the total weight of the terpolymer.

In another embodiment, the shaped article is in the form of a foam structure which includes a copolymer based on the monomers lactide and trimethylene carbonate. The foam structure preferably consists of this copolymer.

The lactide component in the co- and/or terpolymer may be L-lactide, D-lactide and/or DL-lactide, with preference for DL-lactide.

The co- and/or terpolymer may ab initio have a molecular weight in the range from 80 000 to 400 000 daltons, in particular from 90 000 to 250 000 daltons. In the sterilized state, in particular following a gamma irradiation, for example with 25 kGy, the co- and/or terpolymer preferably has a molecular weight of from 50 000 to 150 000 daltons.

The co- and/or terpolymer may additionally have ab initio an inherent viscosity of from 0.8 to 2.5 dl/g, in particular from 1.0 to 2.0 dl/g, based on a 0.1% strength solution of the co- and/or terpolymer in chloroform at 25° C. After a sterilization, in particular after a gamma ray treatment, for example with 25 kGy, the co- and/or terpolymer advantageously has an inherent viscosity of from 0.6 to 1.2 dl/g, based on a 0.1% strength solution of the co- and/or terpolymer in chloroform at 25° C.

In a particularly preferred embodiment of the shaped article of the invention, the foam structure is in the form of a structural foam with a framework which includes voids or cavities. The foam structure, in particular the structural foam, preferably includes a super- and a substructure, where the substructure is formed through the material of the framework itself having a foam structure. The foam structure, in particular in the form of a structural foam, is outstandingly suitable for absorbing wound fluid after being placed on the wound bed.

In a further particularly preferred embodiment of the shaped article of the invention, the superstructure of the foam structure, in particular of the structural foam, has a pore size (pore width) in the range from 50 to 800 μm, in particular in the range greater than 50 to 80 μm, preferably in a range from 80 to 500 μm. The substructure of the foam structure, in particular of the structural foam, is advantageously distinguished by having a pore size in the range from 0.1 to 50 μm, in particular in the range from 0.5 to 30 μm. The pore sizes of the foam structure show in particular an irregular distribution. The distinctly larger pores of the superstructure advantageously have the effect of rapidly and efficiently absorbing the wound fluid or exudate. In this way, infectious material present in the exudate is rapidly removed from the region of the wound, and the wound is thus cleansed. This is particularly advantageous for rapid wound healing.

It is further preferred for the pores of the foam structure, in particular of the structural foam, to be at least partly, preferably completely, connected interconnectingly with one another. The pores are in particular distributed irregularly in the foam structure. The pores may have different shapes. For example, the pores may be rounded pores. A further possibility is for the pores to have an angular form. The total porosity of the shaped article is advantageously from 80 to 96%, in particular 85 to 96%; preferably 90 to 96%. The exceptionally high total porosity of the shaped article of the invention provides a large volume for absorbing wound fluid from the region of the wound. In particular, owing to the large total porosity of the shaped article of the invention, wound fluid flowing back from the region of the wound can also be removed. In addition, a high porosity, especially in combination with optimized pore sizes, allows a vascularization of the shaped article and thus an adequate supply of nutrients and oxygen to the wound site.

In a preferred embodiment, the shaped article has an absorption capacity for liquids which is equivalent to 7 to 15 times, in particular 7 to 12 times, its own weight. The liquids preferably take the form of water or aqueous liquids, especially body and/or tissue fluids. The liquids particularly preferably take the form of wound fluid.

In a further embodiment, the shaped article has a glass transition point (Tg) in the range from 10 to 60° C., preferably in the range from 25 to 37° C. This results in special advantages for use of the shaped article for humans, whose body temperature is in this range.

The shaped article of the invention preferably has a density not exceeding 0.3 g/cm³, preferably less than 0.25 g/cm³, the lower density limit being about 0.05 g/cm³.

It is further preferred according to the invention for the shaped article to be plastically deformable, in particular compressible, extensible and/or bendable. The shaped article of the invention is advantageously, in particular after being placed on a site on the patient's body, increasingly plastically deformable, as a result of the warming to the body temperature thereof, until it reaches an almost free-flowing state. In such a state, the shaped article can adapt to different surface profiles, especially to different areas of the body.

The shaped article may further show at body temperature an extension of from 20 to 150%, the extension being substantially dependent on the proportions of the monomers used in the co- and/or terpolymer. The modulus of elasticity of the shaped article at body temperature is preferably less than 2000 N/mm², preferably less than 1000 N/mm². The elongation at break of the shaped article of the invention likewise depends on the proportions of the monomers used in the co- and/or terpolymer.

The shaped article of the invention is advantageously distinguished by being cuttable, preferably being cuttable at a temperature below the glass transition temperature, preferably below 35° C., of the co- and/or terpolymer.

In a preferred embodiment, the shaped article of the invention has a layer thickness of from 300 μm to 30 cm. The shaped article may according to the invention, in particular after its production, be in the form of a block structure, in particular with a layer thickness of between 5 and 30 cm. The cuttable properties of the shaped article allow it to be provided in the desired cut-out shape depending on the area of application.

Among the possible configurations of the shaped article of the invention, particular preference is given to sheet-like materials, especially flat structures. A shaped article in the form of a sheet-like material, in particular of a flat structure, is preferably distinguished by having a layer thickness of between 300 μm and 5 cm. The shaped article of the invention preferably has a layer thickness in the range above 500 μm, in particular in the range above 500 to 900 μm, preferably in the range from 900 μm to 3 mm. The large layer thickness of the shaped article leads in combination with the large pore width of the superstructure of the foam structure, in particular of the structural foam, to a rapid absorption of large amounts of infectious exudate from the region of the wound and thus to a rapid and effective cleansing of the wound, resulting in particular in speedy wound healing.

It is particularly advantageous for the shaped article of the invention to be distinguished by being completely absorbable in the patient's body, achieving in particular good biocompatibility on use in humans and/or animals. The degradation of the polymer according to the invention takes place in vivo by metabolic processes in which in particular body and tissue fluids are involved. The polymer chain is cleaved by hydrolysis into smaller and in particular more readily soluble fragments. The fragments are further degraded enzymatically where appropriate, and carbon dioxide and water represent the final products. It is further crucial for good biocompatibility that no toxic metabolic intermediates are formed during the degradation process. The monomers used in the shaped article of the invention are distinguished by good compatibility and avoidance of toxic reactions in the body.

It is further advantageous according to the invention for the shaped article to be free of plasticizers, especially for no plasticizer to be added to the co- and/or terpolymer of the shaped article. It is possible in this way to increase further the favorable physiological properties of the shaped article. The flexibility and plastic deformability of the shaped article result from the molecular composition of the co- and/or terpolymer. In particular, a higher content of ε-caprolactone and trimethylene carbonate increases the flexibility of the co- and/or terpolymer. A high lactide content favors the hardness and rigidity of the co- and/or terpolymer.

In a further embodiment, the shaped article of the invention exhibits an in vivo degradation time of from 20 to 35 days. Its absorption time in vivo can be from 70 to 120 days, in particular 80 to 100 days, with the absorption time of the shaped article preferably being complete after 90 days.

During the degradation of the shaped article of the invention, advantageously a slightly acidic environment of about pH 4.5 to 6, in particular of about pH 5, results. This is a physiologically acceptable pH range which corresponds to the conditions of the human body, in particular of the skin surface. This pH range additionally has an antimicrobial, in particular bactericidal, effect and has a wound-stimulating effect, which is advantageous for wound healing.

It is possible according to the invention to provide for the shaped article to have a free monomer content of from 0 to 10% by weight, in particular 0.1 to 10% by weight, preferably 1 to 10% by weight, based on the weight of the co- and/or terpolymer.

In a further embodiment, the co- and/or terpolymer of the shaped article of the invention is doped with a filling material, in which case the filling material forms a lining of at least some of the pores of the foam structure, in particular the pores of the superstructure. The filling material is advantageously in powder form, with the powder preferably having a particle size of from 20 to 500 μm. It is possible in this way to increase the stability of the shaped article effectively. Suitable filling materials are inorganic substances, in particular sodium and/or calcium phosphates, for example tricalcium phosphate (TCP) or hydroxyapatite, with particular preference for tricalcium phosphate. Further suitable filling materials may be natural polymers, in particular proteins, for example collagen. The filling material is preferably a synthetic polymer, with particular preference for polyvinyl alcohol (PVA) in particular because of its very good biocompatibility.

The doping of the shaped article with a filling material is particularly preferred when a strengthening of the structure of the shaped article and a reduction in its extensibility but, at the same time, a rapid and effective absorption of relatively large amounts of exuded fluid from the region of the wound is desired. Thus, in some cases, an extension of from 3 to 10% may be desirable. For this purpose, the shaped article can be doped with a filling material, in particular with tricalcium phosphate (TCP). The co- and/or terpolymer of the shaped article of the invention may have a filling material content in the range between 10 and 300% by weight, preferably between 20 and 100% by weight, based on the total weight of the shaped article.

It is possible to provide in another embodiment for the co- and/or terpolymer to be doped with combinations of filling materials, reference being made to the above description in particular in relation to the filling materials.

The shaped article advantageously comprises medicinally active substances, in particular vaccines, antiseptics, antibiotics, growth factors or the like. Thus, for example, it is particularly preferred for infectious material present in the wound fluid to be killed by antiseptics and/or antibiotics present in the shaped article. This assists and promotes the wound healing process.

The shaped article of the invention may additionally be in particular free of cells or cell cultures.

The invention further includes a process for producing an absorbable and porous shaped article, in particular a shaped article according to the present invention, for medical treatment, in particular as wound-contact material, including the steps:
preparation of a solution of a co- and/or terpolymer in a first solvent,
preparation of a suspension by adding a substance which is insoluble in the first solvent to the solution,
cooling and solidification of the suspension,
precipitation of the co- and/or terpolymer and dissolving out of the substance with a second solvent which is miscible with the first solvent but which is not a solvent for the co- and/or terpolymer.

In one embodiment of the process of the invention, the first solvent used is at least one organic, in particular water-soluble, solvent, where the solvent can preferably be frozen without further difficulties. Suitable solvents used are in particular dimethyl sulfoxide (DMSO), dimethylacetamide (DMA), dimethylformamide (DMF), tetrahydrofuran (THF), dioxane or mixtures thereof, with particular preference for dimethyl sulfoxide (DMSO). Water is preferably used as second solvent. It may be particularly advantageous according to the invention to use dimethyl sulfoxide (DMSO) as first solvent, and water as second solvent, for producing the shaped article.

In a development of the process of the invention, the substance added is a fine-particle substance, preferably solid substance. The substance advantageously has a particle size of from 50 to 500 μm, in particular from 80 to 500 μm, preferably an average particle size of about 200 μm. The substance used is advantageously a hydrophilic or water-soluble substance which dissolves in water or in an aqueous system with minimal energy release (low exothermicity). Suitable substances are in particular salts or organic compounds, for example urea or citric acid.

The substance is preferably sugar. Possible sugars are in particular mono- or disaccharides, for example glucose, fructose, dextrose, maltose, lactose or sucrose. It is further possible to provide according to the invention for the sugars to be polysaccharides, in particular starch, alginates or chitosan. It is possible in a particularly advantageous manner to influence the pore size of the shaped article and thus in particular its absorption capacity for the wound fluid through the choice of the particle size of the substance.

Before, during or after the addition of substance it is possible where appropriate to add a filling material which is insoluble in the second solvent. The filling material is preferably a synthetic polymer, in particular polyvinyl alcohol (PVA). The polyvinyl alcohol (PVA) preferably has a molecular weight of more than 10 000 daltons (10 kDa) so that it is insoluble in water. Reference is made to the previous description for further features of the filling material.

In a further embodiment of the process of the invention, the suspension which is obtained after addition of the substance to the solution of the co- and/or terpolymer and which also includes a filling material where appropriate is solidifed at a temperature of between −10 and −30° C., in particular at a temperature of about −20° C. This is particularly advantageous because embrittlement of the co- and/or terpolymer can be avoided in this way and, in particular, its further processability is facilitated. The precipitation of the co- and/or terpolymer and the dissolving out of the substance is preferably carried out in an aqueous precipitating bath, in particular in one with a water temperature of between 20 and 30° C. This is followed by drying in vacuo. The particles of substance which is soluble in the leaching solvent, in particular water, form, after the dissolving out, the voids of the superstructure of the foam structure, in particular of the structural foam. The pores of the substructure of the foam structure, in particular of the structural foam, are formed during the subsequent drying in vacuo.

The shaped article produced according to the invention can be sterilized with conventional physical and/or chemical methods. One possible sterilization process includes treatment with gamma rays. Another process for sterilizing the shaped article for medical purposes includes the use of ethylene oxide.

The present invention further relates to all shaped articles which are produced or can be produced by the process of the invention, in particular by one of the embodiments described above.

The shaped article of the invention is advantageously suitable for use as wound-covering material in the medical treatment of humans or animals.

In a further preferred embodiment, the shaped article is also suitable as skin substitute material, for example as artificial epidermal and/or dermal skin substitute, in the medical treatment of humans and/or animals, especially for burns, preferably for third degree burns. The shaped article is further suitable for use for adhesion prophylaxis in the medical treatment of humans and/or animals. In a further embodiment, the shaped article is suitable for use as matrix for cell cultures, in particular in the area of regenerative medicine (tissue engineering).

As already mentioned and described, the shaped article of the invention is particularly suitable for the treatment of large-area, deep wounds with heavy discharge, in particular of ulcers and/or deep dermal burn wounds, for example of second and/or third degree burns. A wound environment with exudation is usually a sufficient indication of the presence of an infection. Wounds with heavy exudation, especially ulcers and/or deep dermal burn wounds, impose on those affected a high level of suffering to a particular extent and signify treatment risks which often cannot be anticipated, in particular spread of infections and/or infection with further pathogens. This is associated in particular with a longer stay in hospital and increasing treatment costs. The shaped article of the invention enables, owing to its configuration as foam structure, in particular in combination with the large pore widths of the superstructure and the large layer thickness, large amounts of wound fluid or exudate to be absorbed rapidly, and thus brings about an effective cleansing of the wound. In this way, the risk of spread of infection, and the possibility of contamination with further infectious material in the region of the wound is drastically reduced.

### DESCRIPTION OF THE FIGURES

FIG. 1a-i: SE micrographs of an undoped structural foam made of a terpolymer of the monomers lactide, ε-caprolactone and trimethylene carbonate in four different magnifications,

FIG. 2a-b: SE micrographs of a TCP-doped structural foam made of a copolymer of the monomers lactide and trimethylene carbonate in two different magnifications,

FIG. 3a-b: SE micrographs of a collagen-doped structural foam made of a terpolymer of the monomers lactide, ε-caprolactone and trimethylene carbonate in two different magnifications.

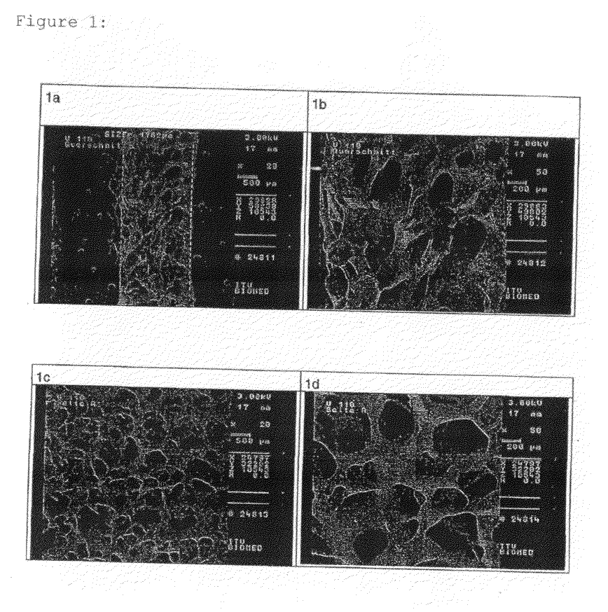

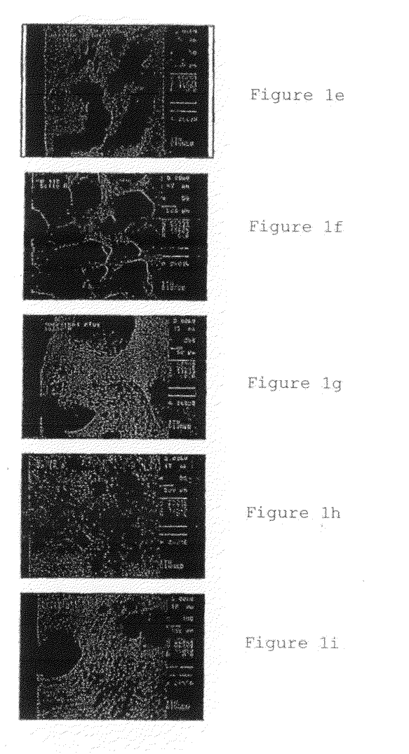

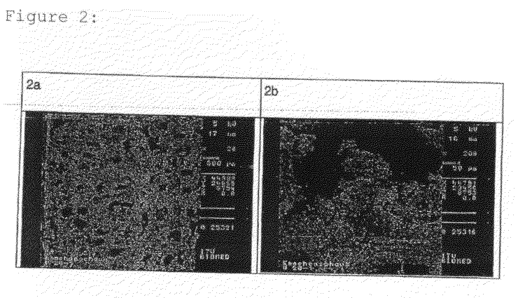

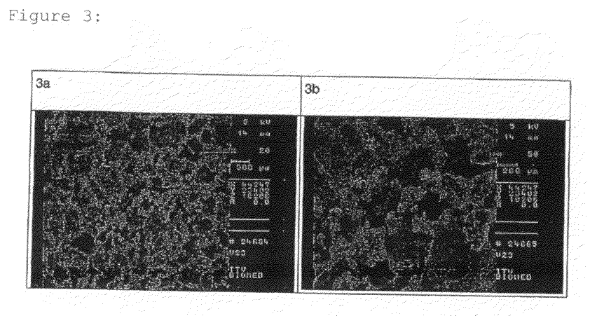

Further features and details of the invention are evident from the following description of preferred embodiments in the form of examples. In this connection, the individual features can in each case be implemented on their own or in combinations of a plurality with one another. The examples serve merely to illustrate the present invention, which is not intended in any way to be restricted thereto.

### EXAMPLE 1

### Preparation of the Terpolymer of Lactide, ε-Caprolactone and Trimethylene Carbonate

To prepare the terpolymer, 1500 g of DL-lactide, 200 g of ε-caprolactone and 300 g of trimethylene carbonate are mixed with stirring. After addition of the catalyst (tin octoate: 0.4 g equivalent to 0.02% by weight based on the total weight of the mixture), the mixture is heated to 180° C. with stirring and polymerized further at this temperature for 24 hours. To draw off the reaction mixture, the temperature is raised to 200° C., and the polymer is discharged and, after cooling, ground to a particle size of 5 mm. The viscosity of the polymer granules is 1.32 dl/g, based on a 0.1% strength solution of the terpolymer in chloroform at 25° C.

### EXAMPLE 2

### Preparation of a Copolymer of Lactide and Trimethylene Carbonate

To prepare the copolymer, 1800 g of DL-lactide and 200 g of trimethylene carbonate are mixed with stirring. After addition of the catalyst (tin octoate: 0.4 g equivalent to 0.02% by weight based on the total weight of the mixture), the mixture is heated to 170° C. with stirring and polymerized further at this temperature for 24 hours. To draw off the reaction mixture, the temperature is raised to 180 to 200° C., and the polymer is discharged and, after cooling, ground to a particle size of 5 mm. The viscosity of the granulated polymer is 1.41 dl/g, based on a 0.1% strength solution of the copolymer in chloroform at 25° C.

### EXAMPLE 3

### Production of Foam without Filling Material

### Batchwise Process

200 g of granulated polymer from example 1 are mixed with 1600 g of dimethyl sulfoxide (DMSO) in a glass reactor and an approx. 11% strength polymer solution is prepared by stirring. 1800 g of sugar (average particle size 200 μm) are added in portions to this solution and stirred vigorously for 5 minutes. The resulting polymer suspension is poured to a height of 5 mm in a steel trough and immediately frozen at −20° C. and left at −20° C. for 2 hours. The frozen solution is put together with the steel trough into a 5 l precipitating bath with double-distilled water at 20° C. to 30° C., it being possible to stir the precipitating bath to speed up the dissolving of the sugar. A marked speeding up of the formation of foam material and of the precipitation and leaching process is achieved with a water circulating apparatus or countercurrent system. The foam material article is then dried to constant weight in vacuo. After the drying, the shaped article can be cut to its desired size, for example with a scalpel or drop knife.

Measured Data:

| | |
|---|---|
| Layer thickness: | 2 mm |
| Porosity: | 90 to 94% |
| Pore size: | 50 to 500 μm, |
| | interconnecting |
| Density: | 0.112 g/cm³ |
| Extension: | 60 to 70% |
| Water absorption capacity: | 1100% (11 times its |
| | own weight) |

### EXAMPLE 4

### Foam Production with TCP as Filling Material

### Batchwise Process

200 g of granulated polymer from example 2 are mixed with 1600 g of dimethyl sulfoxide (DMSO) in a glass reactor and an approx. 11% strength polymer solution is prepared by stirring. 1800 g of sugar and 200 g of tricalcium phosphate powder (TCP powder, average particle size 200 μm) are added in portions to this solution and vigorously stirred for 5 minutes. The resulting polymer suspension is poured to a height of 5 mm in a steel trough and immediately frozen at −20° C. and left at −20° C. for 2 hours. The frozen solution is put together with the steel trough into a 5 l precipitating bath with double-distilled water at 20° C. to 30° C., it being possible to stir the precipitating bath to speed up the dissolving of the sugar.

A marked speeding up of the formation of foam material and of the precipitation and leaching process is achieved with a water circulating apparatus or countercurrent system. The foam material article is then dried to constant weight in vacuo. After the drying, the shaped article can be cut to its desired size, for example with a scalpel or drop knife.

Measured Data:

| | |
|---|---|
| Layer thickness: | 2.5 mm |
| Porosity: | 84 to 88% |
| Pore size: | 30 to 400 μm, |
| | interconnecting |
| Density: | 0.205 g/cm³ |
| Extension: | 3 to 5% |
| Water absorption capacity: | 810% (8 times its |
| | own weight) |

### EXAMPLE 5

### Foam Production with Collagen as Filling Material

### Batchwise Process

200 g of granulated polymer from example 1 are mixed with 1600 g of dimethyl sulfoxide (DMSO) in a glass reactor and an approx. 11% strength polymer solution is prepared by stirring. 1800 g of sugar and 50 g of collagen powder (particle size <50 μm) are added in portions to this solution and vigorously stirred for 5 minutes.

The resulting polymer suspension is poured to a height of 5 mm in a steel trough and immediately frozen at −20° C. and left at −20° C. for 2 hours. The frozen solution is put together with the steel trough into a 5 l precipitating bath with double-distilled water at 20° C. to 30° C., it being advisable to stir the precipitating bath to speed up the dissolving of the sugar.

A marked speeding up of the formation of foam material and of the precipitation and leaching process is achieved with a water circulating apparatus or countercurrent system. The foam material article is then dried to constant weight in vacuo. After the drying, the shaped article can be cut to its desired size, for example with a scalpel or drop knife.

Measured Data:

| | |
|---|---|
| Layer thickness: | 2.5 mm |
| Porosity: | 84 to 88% |
| Pore size: | 30 to 500 μm, |
| | interconnecting |
| Density: | 0.153 g/cm³ |
| Extension: | 40 to 50% |
| Water absorption capacity: | 950% (9.5 times its |
| | own weight) |

1. An absorbable and porous shaped article for the medical treatment of wounds, in particular as wound-contact material, characterized in that it is in the form of a foam structure which includes a co- and/or terpolymer based on the monomers lactide, trimethylene carbonate, ε-caprolactone and/or dioxan-2-one.

2. The shaped article as claimed in claim 1, characterized in that the foam structure is in the form of a structural foam with a framework which includes voids.

3. The shaped article as claimed in claim 2, characterized in that the foam structure includes a superstructure and a substructure, where the substructure is formed through the material of the framework itself having a foam structure.

4. The shaped article as claimed in claim 2, characterized in that the superstructure has a pore size in the range from 50 to 800 μm, in particular in a range greater than 50 to 80 μm, preferably in the range from 80 to 500 μm.

5. The shaped article as claimed in claim 2, characterized in that the substructure has a pore size in the range from 0.1 to 50 μm, in particular in the range from 0.5 to 30 μm.

6. The shaped article as claimed in claim 1, characterized in that it has interconnecting pores.

7. The shaped article as claimed in claim 1, characterized in that it has an absorption capacity for liquids which is equivalent to 7 to 15 times, in particular 8 to 12 times, its own weight.

8. The shaped article as claimed in claim 1, characterized in that it has a density not exceeding 0.3 g/cm³, preferably of less than 0.25 g/cm³.

9. The shaped article as claimed in claim 1, characterized in that it is plastically deformable, in particular compressible, extensible and/or bendable.

10. The shaped article as claimed in claim 1, characterized in that it has a modulus of elasticity of less than 2000 N/mm², preferably of less than 1000 N/mm².

11. The shaped article as claimed in claim 1, characterized in that it is cuttable.

12. The shaped article as claimed in claim 1, characterized in that it has a layer thickness of from 300 μm to 30 cm.

13. The shaped article as claimed in Claim 1, characterized in that it is in the form of a block structure, in particular with a layer thickness of between 5 and 30 cm.

14. The shaped article as claimed in claim 1, characterized in that it is in the form of a sheet-like material, in particular of a flat structure, preferably with a layer thickness of between 300 μm and 5 cm.

15. The shaped article as claimed in claim 1, characterized in that it has a free monomer content of from 0 to 10% by weight, in particular 0.1 to 10% by weight, preferably 1 to 10% by weight, based on the weight of the co- and/or terpolymer.

16. The shaped article as claimed in claim 1, characterized in that the co- and/or terpolymer is doped with a filling material, in particular a synthetic polymer, preferably polyvinyl alcohol (PVA).

17. The shaped article as claimed in claim 16, characterized in that the co- and/or terpolymer has a content of filling material in the range between 10 and 300% by weight, preferably between 20 and 100% by weight, based on the total weight of the shaped article.

18. A process for producing an absorbable and porous shaped article, in particular one as claimed in claim 1, for medical treatment, in particular as wound-contact material, including the steps:
preparation of a solution of a co- and/or terpolymer in a first solvent,
preparation of a suspension by adding a substance which is insoluble in the first solvent to the solution,
cooling and solidification of the suspension,
precipitation of the co- and/or terpolymer and dissolving out of the substance with a second solvent which is miscible with the first solvent but which is not a solvent for the co- and/or terpolymer.

19. The process as claimed in claim 18, characterized in that at least one organic, in particular water-soluble, solvent, preferably dimethyl sulfoxide (DMSO), dimethylacetamide (DMA), dimethylformamide (DMF), tetrahydrofuran (THF), dioxane or mixtures thereof is used as first solvent.

20. The process as claimed in claim 18, characterized in that water is used as second solvent.

21. The process as claimed in claim 18, characterized in that the substance added is a fine-particle substance, preferably solid substance, in particular with a particle size of from 50 to 500 μm, in particular from 80 to 500 μm, preferably with an average particle size of about 200 μm.

22. The process as claimed in claim 18, characterized in that a water-soluble substance, preferably sugar, is used as substance.

23. The process as claimed in claim 18, characterized in that a solid which is insoluble in the second solvent, in particular a synthetic polymer, preferably polyvinyl alcohol (PVA), is added as filling material to the suspension.

24. The process as claimed in claim 18, characterized in that the suspension is solidified at a temperature of between −10 and −30° C., in particular at a temperature of about −20° C.

25. The process as claimed in claim 18, characterized in that the precipitation and dissolving out is carried out in an aqueous precipitating bath, in particular in one with a water temperature of between 20 and 30° C.

26. A shaped article produced by a process as claimed in claim 18.

27. A shaped article which can be produced by a process as claimed in claim 18.

28. The use of a shaped article as claimed in claim 1 as wound-contact material in the medical treatment of humans and/or animals.

29. The use of a shaped article as claimed in claim 1 as skin substitute material in the medical treatment of humans and/or animals.

30. The use of a shaped article as claimed in claim 1 for adhesion prophylaxis in the medical treatment of humans and/or animals.

31. The use of a shaped article as claimed in claim 1 as matrix for cell cultures.
